(19) United States
(12) Patent Application Publication
(10) Pub. No.: US 2005/0043638 A1
(43) Pub. Date: Feb. 24, 2005

(54) Wireless heartbeat-detecting device with electro-magnetic interference shielding device

(76) Inventor: Yu-Yu Chen, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/644,835

(22) Filed: Aug. 21, 2003

(51) Int. Cl. A61B 5/0402

(52) U.S. Cl. 600/509

(57) ABSTRACT

A wireless heartbeat-detecting device with electromagnetic interference shielding device is provided with a shielding member that is mounted via downward extended locating legs thereof on a circuit board to locate above a transmitting antenna by a predetermined distance. A heartbeat signal transmitted from the transmitting antenna is shielded by the shielding member and is sent to a nearby corresponding receiver via only a number of limited signal transmitting paths. Therefore, when a multiple sets of such wireless heartbeat-detecting devices are used at neighboring locations, signals transmitted from the devices do not interfere with one another.

### BACKGROUND OF THE INVENTION

1. Field of the Invention

The present invention relates to a wireless heartbeat-detecting device, and more particularly to a wireless heartbeat-detecting device with an electro-magnetic interference shielding device.

2. Description of the Prior Art

There are various types of exercisers developed for people who live busily in the modern commercial society and require appropriate exercises. And, for a person to accurately control an appropriate quantity of exercise and to monitor personal physical condition, various types of body signal detecting devices have also been researched and developed.

For example, FIG. 1 shows a currently available heartbeat-detecting device that is used to detect a heartbeat or pulse signal from a user 100. This type of heartbeat-detecting device is generally divided into two types, namely, wired and wireless types. Since the wireless type heartbeat-detecting device has the advantages of easy for use and free from limitation by a signal transmitting line, it has been widely used in various places.

A wireless heartbeat-detecting device mainly includes a heartbeat detecting and transmitting unit or detector 101 for fitting on the user's chest, and a receiving and displaying unit or receiver 102 for fitting around the user's wrist. A heartbeat signal detected by the heartbeat detecting and transmitting unit 101 is transmitted via a carrier frequency and then received and displayed by the wrist-type receiving and displaying unit 102.

The heartbeat detecting and transmitting unit 101 of the wireless heartbeat-detecting device basically includes a conductive rubber pad serving as an electrode to detect a heartbeat signal, and a circuit board for processing and transmitting signals. When the conductive rubber pad and the circuit board are electrically connected, an actual heartbeat signal detected by the conductive rubber pad may be wirelessly transmitted via a signal transmitter of the circuit board.

FIG. 2 shows a transmitting antenna 103 for the conventional wireless heartbeat-detecting device that is not shielded with any means. In this condition, a heartbeat pulse signal in a form of electromagnetic wave transmitted out at both ends of the transmitting antenna 103. In a most ideal condition, only a nearby corresponding receiver is able to receive the heartbeat pulse signal transmitted from the signal transmitter of the wireless heartbeat-detecting device. However, in the use of such wireless heartbeat-detecting device, the problem of signal interference always happens. For example, when the conventional wireless heartbeat-detecting device is used in some places, such as in a health club, a gymnasium, or a rehabilitation center, it is frequently interfered by various signals from other wireless heartbeat-detecting devices because the distances among the users are too close. Thus, it is likely that the heartbeat signal is not received probably and data about the heartbeat signal is incorrectly determined.

A practice commonly and conventionally adopted to prevent the signal interference among neighboring wireless heartbeat-detecting devices is to include a signal encoding and decoding circuit in the transmitter and the receiver of the heartbeat-detecting devices. There are also other products that solve the problem of signal interference by way of using different radio channels to send the wireless heartbeat signals. However, this design does not effectively solve the problem of signal interference in the actual use thereof.

Therefore, it is necessary to take additional measures to effectively overcome the problem of signal interference to meet user's actual need.

### SUMMARY OF THE INVENTION

A primary object of the present invention is to provide an interference shielding structure for a wireless heartbeat-detecting device, so that even though a plurality of wireless heartbeat-detecting devices are used at close distances, interference among the wireless heartbeat-detecting devices is avoided.

Another object of the present invention is to provide a wireless heartbeat-detecting device with an electromagnetic interference shielding device. The electromagnetic interference shielding device is simple in structure and enables neighboring persons to use the wireless heartbeat-detecting devices without signal interference.

To achieve the above and other objects, the wireless heartbeat-detecting device according to an embodiment of the present invention includes a shielding member which is positioned above a transmitting antenna by a predetermined distance by fastening two downward extended locating legs at the two corresponding ends of the shielding member on a circuit board of the wireless heartbeat-detecting device, so that a heartbeat signal transmitted from the transmitting antenna is shielded by the electromagnetic interference shielding member and is sent to a nearby corresponding receiver via only limited signal transmitting paths. Therefore, when multiple sets of such wireless heartbeat-detecting devices are used at neighboring region, signals transmitted from the devices would not interfere with one another.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 schematically shows a conventional wireless heartbeat-detecting device fitted on a user's body;

FIG. 2 schematically shows a transmitting antenna of the conventional wireless heartbeat-detecting device with no shielding, in which a heartbeat pulse signal in a form of electromagnetic wave transmitted out at both ends of the antenna;

FIG. 3 is a partially exploded perspective view of a wireless heartbeat-detecting device with electromagnetic interference shielding device according to a first embodiment of the present invention;

FIG. 4 is a fully exploded perspective view of the wireless heartbeat-detecting device with electromagnetic interference shielding device of FIG. 3;

FIG. 5 is a sectional view taken along line s of FIG. 3;

FIG. 6 is a schematic view showing a transmitting antenna of the wireless heartbeat-detecting device with electromagnetic interference shielding device, in which the transmitting antenna is shielded by a shielding member of the present invention and a heartbeat signal in the form electro-magnectic wave transmitted at two ends of the transmitting antenna is attenuated;

FIG. 7 schematically shows that a plurality of signals transmitted from a multiple sets of the wireless heartbeat-detecting device with electro-magnetic interference shielding device of the present invention at close distances do not interfere with one another;

FIG. 8 is a perspective view of a shielding member according to a second embodiment of the present invention; and

FIG. 9 is a perspective view of a shielding member according to a third embodiment of the present invention.

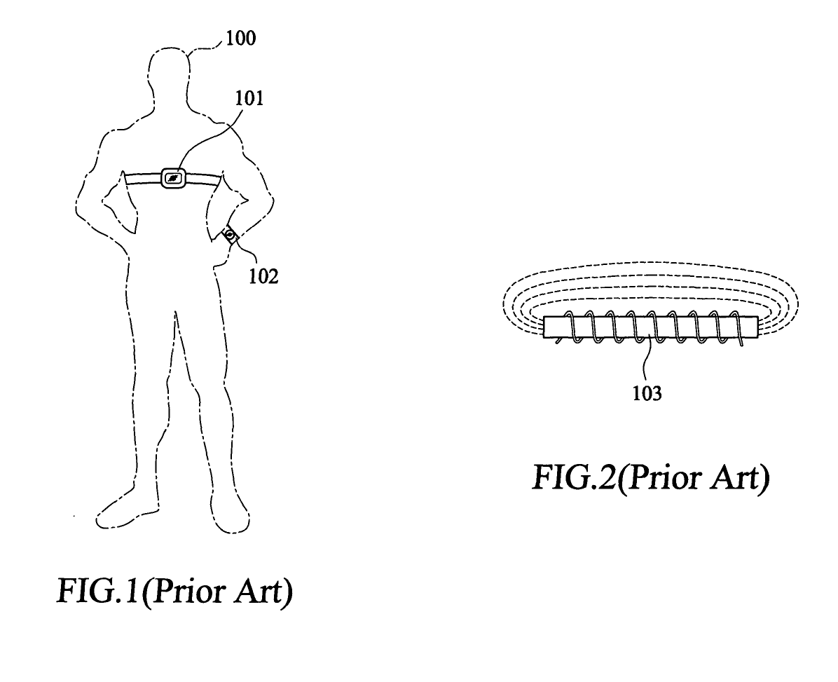

### DETAILED DESCRIPTION OF THE PREFERRED EMBODIMENTS

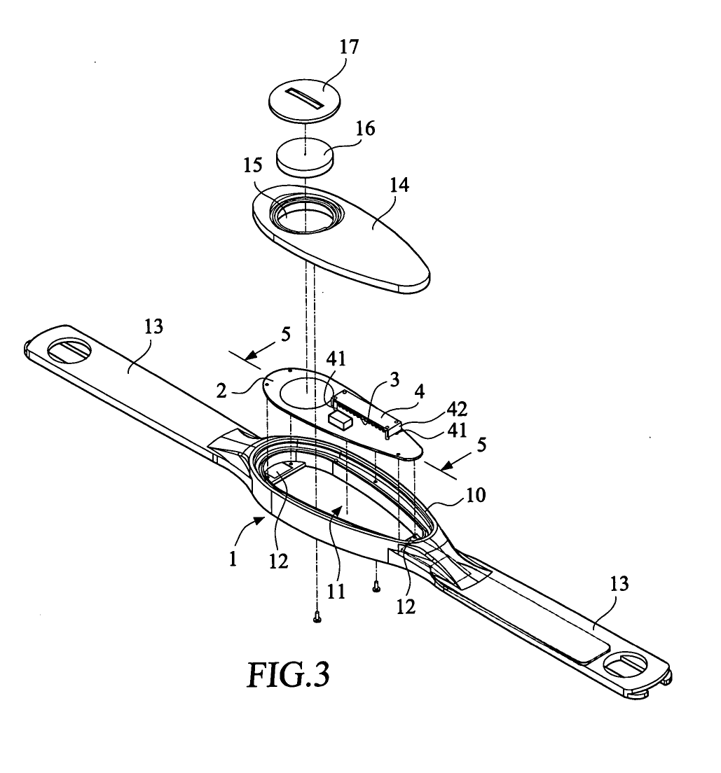

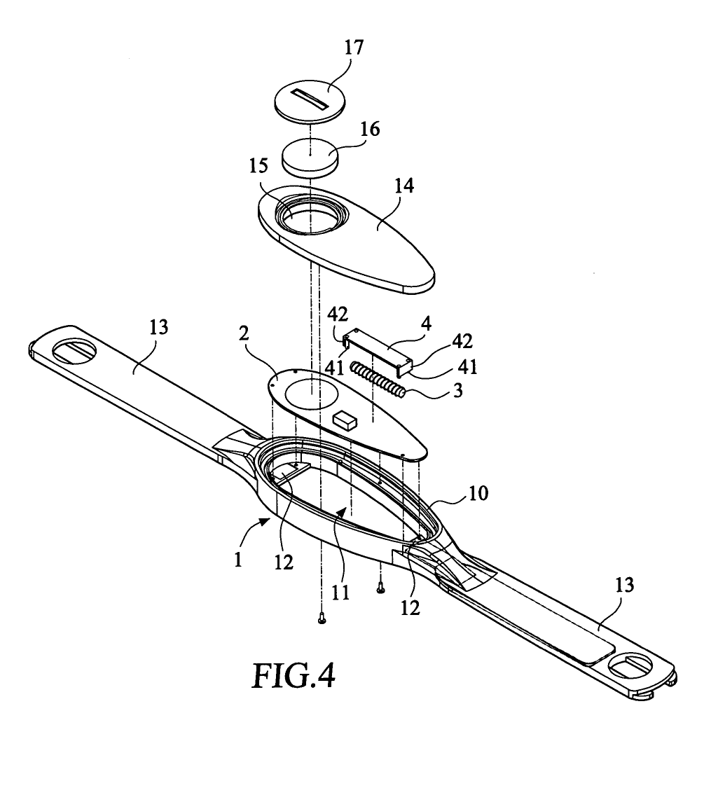

Please refer to FIGS. 3 and 4 that are partially and fully exploded perspective views, respectively, of a wireless heartbeat-detecting device with an electromagnetic interference (EMI) shielding device according to a first preferred embodiment of the present invention. For the purpose of clarity, the present invention is referred to as the wireless heartbeat-detecting device and generally denoted by reference number 1. As shown, the wireless heartbeat-detecting device 1 mainly includes a belt-associated seat 10 defining an internal space 11. Two conductive sections 12 are separately provided at two lateral ends of the internal space 11, and two belts 13 are separately extended from an outer side of the two conductive sections 12. The two belts 13 may be tied to a user's trunk to locate the wireless heartbeat-detecting device 1 at the user's chest.

A circuit board 2 is mounted in the internal space 11 of the belt-associated seat 10. A conventional control circuit is provided on the circuit board 2 and includes a transmitting antenna 3 that is disposed on the circuit board 2 and connected to a wireless signal transmitting circuit of the control circuit of the wireless heartbeat-detecting device 1, so that a heartbeat signal detected by the wireless heartbeat-detecting device 1 may be transmitted via the wireless signal transmitting circuit.

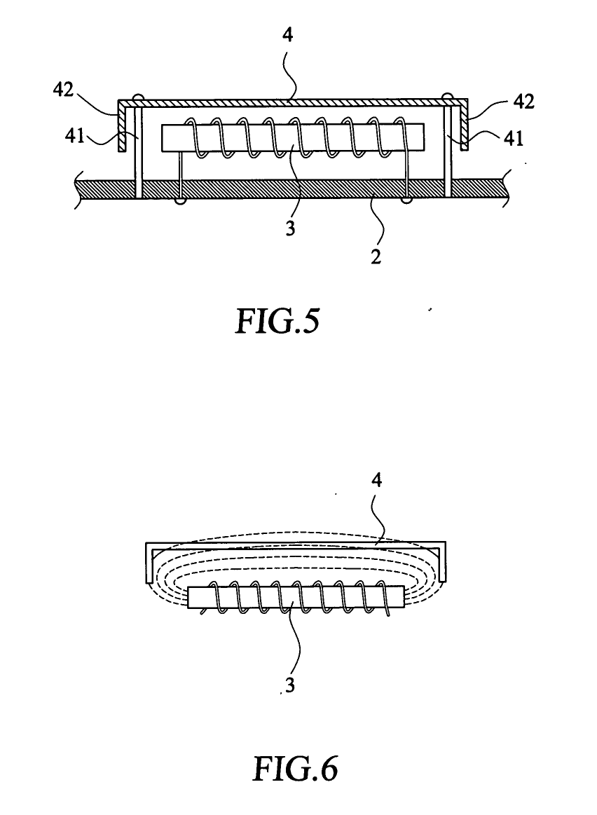

In the embodiment, the circuit board 2 also includes a shielding member 4 which is mounted at a predetermined distance above the transmitting antenna 3, as can be clearly seen from FIG. 5. With the shielding member 4, the heartbeat signal transmitted via the transmitting antenna 3 is shielded at certain directions and areas and therefore can only be transmitted along restricted transmitting paths to a nearby corresponding receiver. FIG. 6 shows that when the transmitting antenna 3 is housed in the shielding member 4, the transmitted heartbeat signal in the form of electromagnetic wave at two lateral ends of the transmitting antenna 3 is attenuated.

The shielding member 4 is a substantially flat plate having two downward extended wing portions 42 provided at two lateral ends thereof. A plurality of locating legs 41 are extended from the shielding member 4 toward the circuit board 2. By engaging the locating legs 41 to the circuit board 2, the shielding member 4 is positioned above the transmitting antenna 3 by a predetermined distance. Moreover, the locating legs 41 of the shielding member 4 are connected to a grounding conductor of the circuit board 2.

A cover 14 is provided at a top of the belt-associated seat 10 which comprises a battery compartment 15 for holding a battery 16 therein. The battery compartment 15 is then openably closed with a lid 17.

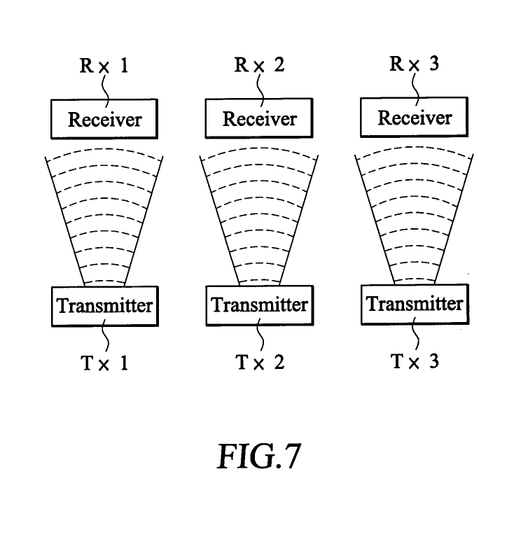

FIG. 7 shows that a multiple sets of wireless heartbeat-detecting devices 1 of the present invention are used by closely located users at the same time. Signals from transmitters TX1, TX2, and TX3 of these multiple sets of wireless heartbeat-detecting devices 1 are shielded by respective shielding members 4 and is only capable to transmit to corresponding receivers RX1, RX2, and RX3 of the multiple sets of wireless heartbeat-detecting devices 1. In other words, receivers RX1, RX2, and RX3 are not able to receive signals from any nearby transmitters except the sole corresponding transmitter. Thereby mutual interference is avoided.

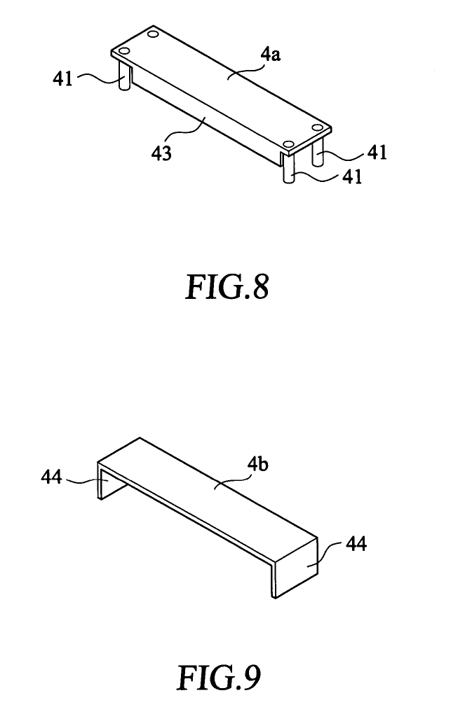

FIG. 8 shows a shielding member 4a according to a second embodiment of the present invention. As shown, the shielding member 4a includes two wing portions 43 formed at two opposite longitudinal sides thereof. With the wing portions 43 having an extended length, transmitted directions and areas of signals transmitted via the transmitting antenna 3 may be further limited.

FIG. 9 shows a shielding member 4b according to a third embodiment of the present invention. As shown, the shielding member 4b is a substantially flat plate having two wing portions 44 formed at two lateral ends thereof. The wing portions 44 are fastened to the circuit board 2 and completely shield the heartbeat signal transmitting at two lateral ends of the transmitting antenna 3.

From the preferred embodiments, it is apparent that shielding device of the present invention effectively shields the signal of a wireless heartbeat-detecting device and prevents interfering nearby wireless heartbeat-detecting devices.

The present invention has been described with some preferred embodiments thereof and it is understood that many changes and modifications in the described embodiments can be carried out without departing from the scope and the spirit of the invention that is intended to be limited only by the appended claims.

1. A wireless heartbeat-detecting device with an electromagnetic interference shielding device, comprising:
a belt-associated seat for detecting a heartbeat pulse signal from a user and defining an internal space;
a circuit board seated in the internal space of the belt-associated seat, and having a heartbeat detecting circuit for processing the heart beat signal detected by the belt-associated seat and a wireless signal transmitting circuit provided thereon;
a transmitting antenna mounted on the circuit board and connected to the wireless signal transmitting circuit for transmitting a series of heartbeat signals processed and transmitted by the heartbeat detecting circuit; and
a shielding member mounted on the circuit board at a position at where the transmitting antenna is located, and a predetermined spacing height being kept between the shielding member and the transmitting antenna, so that the heartbeat signal transmitted by the transmitting antenna is shielded and is sent to a nearby corresponding receiver only via a limited number of signal transmitting paths.

2. The wireless heartbeat-detecting device as claimed in claim 1, wherein the shielding member is located above the transmitting antenna, and connected to a grounding conductor of the circuit board.

3. The wireless heartbeat-detecting device as claimed in claim 1, wherein the shielding member is a substantially flat plate member.

4. The wireless heartbeat-detecting device as claimed in claim 1, wherein the shielding member has at least two downward extended wing portions at two corresponding ends of the shielding member.
